(19) United States
(12) Patent Application Publication
(10) Pub. No.: US 2003/0096000 A1
(43) Pub. Date: May 22, 2003

(54) Encapsulation of nanosuspensions in liposomes and microspheres

(76) Inventors: Rosa Maria Solis, San Diego, CA (US); Sankaram Mantripragada, San Diego, CA (US); Pascal Grenier, Kappelen (FR); Alain Nhamias, Bartenheim (FR)

Correspondence Address: DIANE L. GARDNER, Fish & Richardson P.C., Suite 500, 4350 La Jolla Village Drive, San Diego, CA, 92122, US

(21) Appl. No.: 10/161,969

(22) Filed: May 31, 2002

Related U.S. Application Data
(60) Provisional application No. 60/295,233, filed on May 31, 2001.

(51) Int. Cl. A61K 9/127

(52) U.S. Cl. 424/450

(57) ABSTRACT

Sustained release of hydrophobic agents may be achieved by incorporation of the agents into liposomes and microspheres. This is achieved by use of a nanosuspension comprising the hydrophobic agent. The nanosuspension may be used as the aqueous solution in the formation of the liposomes and microspheres.

### CROSS-REFERENCE TO RELATED APPLICATIONS

This application claims priority under 35 U.S.C. §119(e)(1) to U.S. Patent Application No. 60/295,233, filed May 31, 2001.

### BACKGROUND

Nanoparticle technology expands diagnostic and therapeutic delivery capabilities by enabling preparation of sparingly soluble or insoluble hydrophobic agents as aqueous suspensions containing liquid and/or solid particles in the nanometer size range. The small particle size results in large surface area, which increases the rate of dissolution, directly affecting the bioavailability of the agents. The resulting particle-containing suspensions are typically referred to as “nanosuspensions.”

Liposomes are synthetic, single or multi-compartmental vesicles having lipid or lipid/polymer membranes enclosing aqueous chambers. It is to be understood that wherever the term “lipid” is used herein, it also includes “lipid/polymer” as an alternative. There are at least three types of liposomes. “Multilamellar liposomes or vesicles (MLV)” have multiple “onion-skin” concentric lipid membranes, in between which are shell-like concentric aqueous compartments. “Unilamellar liposomes or vesicles (ULV)” refers to liposomal structures having a single aqueous chamber. “Multivesicular liposomes (MVL)” are lipid vesicles comprising lipid membranes enclosing multiple, non-concentric aqueous compartments.

Microspheres are particles having an outer membrane comprised of synthetic or natural polymers surrounding an aqueous chamber. They are generally discrete units that do not share membranes when in suspension.

Generally, water-soluble agents are incorporated into liposomes and microspheres because the internal compartments are aqueous. Incorporation of sparingly soluble or insoluble agents into liposomes can be accomplished by a method that introduces the hydrophobic agents into the solvent phase during synthesis, thereby resulting in the presence of the agents in the lipid bi-layer of the liposomes.

Until now, nanosuspension, liposome and microsphere technologies have been considered as separate delivery systems.

### SUMMARY

Sustained release of hydrophobic agents may be achieved by incorporation of the agents into the chambers of liposomes and microspheres. This is achieved by use of a nanosuspension comprising the hydrophobic agent. The nanosuspension may be used as the aqueous phase in the formation of the liposomes and microspheres. The liposome membranes may be lipid membranes or they may be comprised of lipid/polymer combinations. Alternatively, microspheres may be made wherein the membranes are composed of synthetic and/or natural polymers.

### BRIEF DESCRIPTION OF THE DRAWINGS

These and other aspects will now be described in detail with reference to the accompanying drawings, wherein:

FIG. 1 shows a laser diffractometry diagram of particle size distribution for a parent glibenclamide suspension prior to homogenization;

FIG. 2 shows a photon correlation spectroscopy diagram of particle size distribution for a glibenclamide nanosuspension;

FIG. 3 shows a laser diffractometry diagram of particle size distribution for a parent nifedipine suspension prior to homogenization;

FIG. 4 shows a photon correlation spectroscopy diagram of particle size distribution for a nifedipine nanosuspension;

FIG. 5 shows percent encapsulated and percent unencapsulated glibenclamide for three batches of glibenclamide nanosuspensions encapsulated in multivesicular liposomes;

FIG. 6 shows percent encapsulated and percent unencapsulated glibenclamide for three batches of glibenclamide nanosuspensions encapsulated in multivesicular liposomes;

FIG. 7 shows percent loading for three batches of glibenclamide nanosuspensions encapsulated in multivesicular liposomes;

FIG. 8 shows percent packed particle volume (lipocrit) for three batches of glibenclamide nanosuspensions encapsulated in multivesicular liposomes;

FIGS. 9 and 10 show micrographs comparing blank multivesicular liposomes (FIG. 9) and multivesicular liposomes containing 5% anhydrous dextrose, Tween® 80, and polyvinyl pyrrolidone (PVP) in the first aqueous phase (FIG. 10);

FIG. 11 shows a comparison of the effects of Tween® 80 and PVP on multivesicular liposome particle size;

FIG. 12 shows a comparison of the effects of Tween® 80 and PVP on percent lipocrit;

FIG. 13 shows a comparison of multivesicular liposome-nanosuspension (MVL-NS)formulations using various solvents;

FIG. 14 shows a micrograph of multivesicular liposomes made with Forane® 141B;

FIG. 15 shows micrograph of MVL-NS made with Forane® 141B;

FIG. 16 shows micrograph of MVL-NS made with isopropyl ether;

FIG. 17 shows micrograph of MVL-NS made with 1,1,1-trichloroethane;

FIG. 18 shows a micrograph (width=12.5 μm) of a blank multivesicular liposome;

FIG. 19 shows a micrograph (width=3.3 μm) of a nanosuspension (mean particle size=600 nm);

FIG. 20 shows a micrograph (width=4.6 μm) of a multivesicular liposome encapsulating a nanosuspension (mean particle size=360 nm);

FIG. 21 shows a micrograph (width=7.8 μm) of a multivesicular liposome encapsulating a nanosuspension (mean particle size=600 nm);

FIG. 22 shows in vitro release rates of multivesicular liposome-encapsulated perphenazine solution and multivesicular liposome-encapsulated perphenazine nanosuspension; and

FIG. 23 shows a pharmacokinetic comparison of perphenazine solution, perphenazine nanosuspension and multivesicular liposome encapsulated perphenazine solution.

### DETAILED DESCRIPTION

Nanosuspensions

Nanosuspensions (NS) and various methods for making them are well known in the art. As used herein, the term “nanosuspension” means any aqueous suspension containing liquid and/or solid particles ranging in size approximately from nanometer to micron. The nanosuspension contains the hydrophobic particles for incorporation into the liposomes and microspheres. This invention is not limited by specific types of nanosuspensions. Any nanosuspension may be employed, as further described herein, it being understood that each resulting liposome-nanosuspension or microsphere-nanosuspension formulation should be prepared appropriately for the desired route of administration (e.g., topical, inhalation, oral, and parenteral). Other conventional considerations also should be contemplated, such as the use of biocompatible ingredients and agent concentration appropriate for the particular use desired. These factors are easily recognized and can be suitably determined by any person having ordinary skill in the art.

Nanosuspensions prepared by any method may be used according to the invention. For example, nanosuspensions may be prepared by mixing solvent and non-solvent in a static blender and fast-mixing in order to obtain a highly dispersed product. Nanosuspensions also may be prepared by various milling techniques. For example, use of jet mills, colloid mills, ball mills and pearl mills are all well known in the art. Detailed descriptions of these processes can be found, for example, in The Handbook of Controlled Release Technology edited by Donald L. Wise (Marcel Dekker, 2000).

Another method for preparing nanosuspensions is via hot or cold high-pressure homogenization, e.g., through use of a piston gap homogenizer or microfluidizer. It should be understood that the foregoing methods of preparation are provided merely as examples of well-known processes, and are not to be considered all-inclusive of the types of methods that may be employed for the preparation of nanosuspensions.

The nanosuspensions may be stabilized with use of a wide variety of surface modifiers or surfactants, and also may contain polymers, lipids and/or excipients. Nanosuspensions may be preserved for later use, e.g., via freeze-drying, spray-drying or lyophilization. Where surfactants are employed, they may be selected based upon criteria well-known in the art, such as quantity and rapidity of water uptake, determination of critical micellar concentration (CMC), and adsorption isotherms. Agents

The particular agent in the nanosuspension is not limited to any particular category. “Agent” means a natural, synthetic or genetically engineered chemical or biological compound having utility for interacting with or modulating physiological processes in order to afford diagnosis of, prophylaxis against, or treatment of, an existing or pre-existing condition in a living being. Agents additionally may be bi- or multi-functional.

Agents in nanosuspensions are hydrophobic, sparingly soluble or insoluble in water. Examples of useful agents include, but are not limited to antineoplastics, blood products, biological response modifiers, anti-fungals, antibiotics, hormones, vitamins, peptides, enzymes, dyes, anti-allergics, anti-coagulants, circulatory agents, metabolic potentiators, antituberculars, antivirals, antianginals, anti-inflammatories, antiprotozoans, antirheumatics, narcotics, opiates, diagnostic imaging agents, cardiac glycosides, neuromuscular blockers, sedatives, anesthetics, as well as magnetic, paramagnetic and radioactive particles. Other biologically active substances may include, but are not limited to monoclonal or other antibodies, natural or synthetic genetic material, proteins, polymers and prodrugs.

As used herein, the term “genetic material” refers generally to nucleotides and polynucleotides, including nucleic acids such as RNA and DNA of either natural or synthetic origin, including recombinant, sense and antisense RNA and DNA. Types of genetic material may include, for example, nucleic acids carried on vectors such as plasmids, phagemids, cosmids, yeast artificial chromosomes, and defective (helper) viruses, antisense nucleic acids, both single and double stranded RNA and DNA and analogs thereof.

Typically, nanosuspensions having smaller particle sizes in the nanometer ranges result in greater yields, as measured by the final concentration of the agent in the resulting liposome-nanosuspension or microsphere-nanosuspension formulations. Some agents, however, require only small yields for effectiveness. Therefore, particle sizes in the micro ranges also may be utilized effectively. A person having ordinary skill in the art can determine the appropriate yield and particle sizes required for effectiveness for any given agent in view of the desired use.

Due to the sizes and nature of the particles in nanosuspensions, liposomes and microspheres having internal chambers of about 1 μm diameter or greater are useful for encapsulation of the agents in the nanosuspensions. The agent may or may not be present in suspension within the resulting internal chambers. In particular, multivesicular liposomes are useful because of their multiple internal chambers in the 1-3 μm range.

Liposomes

Methods of producing liposomes are well known in the art. For example, well-known methods of liposome production include, but are not limited to, hydration of dried lipids, solvent or detergent removal, reverse phase evaporation, sparging, double emulsion preparation, fusion, freeze-thawing, lyophilization, electric field application, and interdigitation-fusion. Detailed descriptions of these processes may be found, for example, in Liposomes—Rational Design edited by Andrew S. Janoff (Marcel Dekker, 1999). Other processes for preparation of liposomes can be found in the art. See, for example, co-pending U.S. application Ser. No. 09/192,064. The foregoing list provides mere examples of various methods of producing liposomes. Various other methods that may be employed for producing liposomes are well-known in the art.

In addition to the particle size and particular method steps employed, other factors, such as the types of lipids and polymers used, the degree of unsaturation and the membrane surface charge, may all affect the resulting yield. Multivesicular liposomes made by the double emulsion process are particularly useful. This method is described in U.S. Pat. No. 6,132,766.

The lipids used may be natural or synthetic in origin and include, but are not limited to, phospholipids, sphingolipids, sphingophospholipids, sterols and glycerides. The lipids to be used in the compositions of the invention are generally amphipathic, meaning that they have a hydrophilic head group and a hydrophobic tail group, and may have membrane-forming capability. The phospholipids and sphingolipids may be anionic, cationic, nonionic, acidic or zwitterionic (having no net charge at their isoelectric point), wherein the hydrocarbon chains of the lipids are typically between 12 and 22 carbons atoms in length, and have varying degrees of unsaturation.

Useful anionic phospholipids include phosphatidic acids, phosphatidylserines, phosphatidylglycerols, phosphatidylinositols and cardiolipins. Useful zwitterionic phospholipids are phosphatidylcholines, phosphatidylethanolamines and sphingomyelins. Useful cationic lipids are diacyl dimethylammonium propanes, acyl trimethylammonium propanes, and stearylamine. Useful sterols are cholesterol, ergosterol, nanosterol, or esters thereof.

The glycerides can be monoglycerides, diglycerides or triglycerides including triolein, and can have varying degrees of unsaturation, with the fatty acid hydrocarbon chains of the glycerides typically having a length between 4 and 22 carbons atoms. Combinations of these lipids also can be used. The choice of lipid or lipid combination will depend upon the desired method for liposome production and the interplay between the liposome components and the agent in nanosuspension, as well as the desired encapsulation efficiency and release rate, as described herein. The liposomes additionally may be coated with polymers.

Lipid/polymer Liposomes and Polymeric Microspheres

Lipid/polymer liposomes and polymeric microspheres are known in the art. A method of producing such lipid/polymer liposomes is described, for example, in U.S. application Ser. No. 09/356,218. Methods of producing microspheres are described, for example, in U.S. Pat. Nos. 5,552,133, 5,310,540, 4,718,433 and 4,572,203; European Patent Publication No. EP 458,745; and PCT Publication No. WO 92/05806. Where a biodegradable polymer is employed in the membrane of the liposome or microsphere, the biodegradable polymer may be a homopolymer, or a random or block copolymer, or a blend or physical mixture thereof. Unless the optical activity of a particular material is designated by [L]- or [D]-, the material is presumed to be achiral or a racemic mixture. Meso compounds (those compounds with internally canceling optical activity) are also useful in the present invention.

A biodegradable polymer is one that can be degraded to a low molecular weight and may or may not be eliminated from a living organism. The products of biodegradation may be the individual monomer units, groups of monomer units, molecular entities smaller than individual monomer units, or combinations of such products. Such polymers also can be metabolized by organisms. Biodegradable polymers can be made up of biodegradable monomer units. A biodegradable compound is one that can be acted upon biochemically by living cells or organisms, or parts of these systems, or reagents commonly found in such cells, organisms, or systems, including water, and broken down into lower molecular weight products. An organism can play an active or passive role in such processes.

The biodegradable polymer chains useful in the invention preferably have molecular weights in the range 500 to 5,000,000 Da. The biodegradable polymers can be homopolymers, or random or block copolymers. The copolymer can be a random copolymer containing a random number of subunits of a first copolymer interspersed by a random number of subunits of a second copolymer. The copolymer also can be block copolymer containing one or more blocks of a first copolymer interspersed by blocks of a second copolymer. The block copolymer also can include a block of a first copolymer connected to a block of a second copolymer, without significant interdispersion of the first and second copolymers.

Biodegradable homopolymers useful in the invention can be made up of monomer units selected from the following groups: hydroxy carboxylic acids such as α-hydroxy carboxylic acids including lactic acid, glycolic acid, lactide (intermolecularly esterified dilactic acid), and glycolide (intermolecularly esterified diglycolic acid); β-hydroxy carboxylic acids including β-methyl-β-propiolactone; γ-hydroxy carboxylic acids; δ-hydroxy carboxylic acids; and ε-hydroxy carboxylic acids including ε-hydroxy caproic acid; lactones such as: β-lactones; γ-lactones; δ-lactones including valerolactone; and ε-lactones such as ε-caprolactone; benzyl ester-protected lactones such as benzyl malolactone; lactams such as: β-lactams; γ-lactams; δ-lactams; and ε-lactams; thiolactones such as 1,4-dithiane-2,5-dione; dioxanones; unfunctionalized cyclic carbonates such as: trimethylene carbonate, alkyl substituted trimethylene carbonates, and spiro-bis-dimethylene carbonate (2,4,7,9-tetraoxa-spiro[5.5]undecan-3,8-dione); anhydrides; substituted N-carboxy anhydrides; propylene fumarates; orthoesters; phosphate esters; phosphazenes; alkylcyanoacrylates; aminoacids; polyhydroxybutyrates; and substituted variations of the above monomers.

The use of such monomers results in homopolymers such as polylactide, polyglycolide, poly(p-dioxanone), polycaprolactone, polyhydroxyalkanoate, polypropylenefumarate, polyorthoesters, polyphosphate esters, polyanhydrides, polyphosphazenes, polyalkylcyanoacrylates, polypeptides, or genetically engineered polymers, and other homopolymers which can be formed from the above mentioned examples of monomers. Combinations of these homopolymers also can be used to prepare the microspheres of the pharmaceutical compositions of the invention.

The biodegradable copolymers can be selected from poly(lactide-glycolide), poly(p-dioxanone-lactide), poly(p-dioxanone-glycolide), poly(p-dioxanone-lactide-glycolide), poly(p-dioxanone-caprolactone), poly(p-dioxanone-alkylene carbonate), poly(p-dioxanone-alkylene oxide), poly(p-dioxanone-carbonate-glycolide), poly(p-dioxanone-carbonate), poly(caprolactone-lactide), poly(caprolactone-glycolide), poly(hydroxyalkanoate), poly(propylenefumarate), poly(ortho esters), poly(ether-ester), poly(ester-amide), poly(ester-urethane), polyphosphate esters, polyanhydrides, poly(ester-anhydride), polyphospazenes, polypeptides or genetically engineered polymers. Combinations of these copolymers also can be used to prepare the microspheres of the pharmaceutical compositions of the invention.

Useful biodegradable polymers are polylactide, and poly(lactide-glycolide). In some lactide-containing embodiments, the polymer is prepared by polymerization of a composition including lactide in which greater than about 50% by weight of the lactide is optically active and less than 50% is optically inactive, i.e., racemic [D,L]-lactide and meso [D,L]-lactide. In other embodiments, the optical activity of the lactide monomers is defined as [L], and the lactide monomers are at least about 90% optically active [L]-lactide. In still other embodiments, the lactide monomers are at least about 95% optically active [L]-lactide.

The foregoing merely exemplifies various methods of producing lipid/polymer liposomes and microspheres. Various other methods that may be employed for producing lipid/polymer liposomes and microspheres are well-known in the art.

Solvents

When the method of preparation of the liposome or microsphere requires a solvent, the types of solvents that are useful are determined by their inability to dissolve the drug crystals in the nanosuspensions while still being capable of dissolving the lipids and polymers present in the membranes of the liposomes and microspheres. Other factors, obvious to any person having ordinary skill in the art, include considerations such as biocompatibility. Proper solvents for use with particular agents and liposome or microsphere formulations may be determined through routine experimentation by any person having ordinary skill in the art.

General Method of Preparation

Typically, the nanosuspensions are encapsulated within the liposome or microsphere chambers by using the nanosuspension as the aqueous phase during liposome or microsphere formation process. Proper concentrations of the agent in the nanosuspension will depend upon the desired use for the resulting composition and may be easily determined by any person having ordinary skill in the art. The resulting particles may have the agent situated within the vesicles or associated on the surface. An excess of agent on the surface of the particles may be washed away. The agent also may be present within the membranes of the resulting liposomes, lipid/polymer liposomes or microparticles.

The agents may be used alone or in combination, either together in the starting nanosuspension, or in separate nanosuspensions encapsulated in separate chambers within multi-chambered particles, such as multivesicular liposomes. The amount of the agent(s) in the final composition should be sufficient to enable the diagnosis of, prophylaxis against, or the treatment of, an existing or pre-existing condition in a living being. Generally, the dosage will vary with the age, condition, sex, and extent of the condition in the patient, and can be determined by one skilled in the art. The dosage range appropriate for human use includes a range of 0.1 to 6,000 mg of the agent per square meter of body surface area.

Other process parameters for adjusting the yield or the characteristics of the liposomes and microspheres are known in the art and may be employed. For example, it is known that heterovesicular liposomes may be produced wherein more than one agent is encapsulated separately in the chambers of multivesicular liposomes. This process is described, for example, in U.S. Pat. No. 5,422,120. In this process, multiple “first” aqueous phases are employed in sequence for each of the separately encapsulated agents.

It is also known that the release rate of the agents from liposomes may be controlled by adjusting the osmolarity of the aqueous phase. This process is described, for example, in U.S. Pat. No. 5,993,850. Complexing the agent with cyclodextrin also may modify the release rate. This process is described, for example, in U.S. Pat. No. 5,759,573. In emulsion processes for making liposomes, agent release rate also may be adjusted by altering acid concentration in the water-in-oil emulsion. See, for example, U.S. Pat. No. 5,807,572. Moreover, the ratio of slow release neutral lipids to fast release neutral lipids, when used in conjunction with amphipathic lipids, may additionally modify the release rate of agents from liposomes. This process is described, for example, in U.S. Pat. No. 5,962,016.

It is further known that modification of the number of carbons in the fatty acyl chain of an amphipathic lipid used to produce liposomes (e.g., U.S. Pat. No. 5,997, 899) and/or modification of the osmolarity of the aqueous phase can modify the percent of the agent encapsulated within the vesicles. Osmotic excipients useful for this purpose include, but are not limited to glucose, sucrose, trehalose, succinate, glycylglycine, glucuronic acid, arginine, galactose, mannose, maltose, mannitol, glysine, lysine, citrate, sorbitol dextran and suitable combinations thereof. See, for example, U.S. Pat. No. 6,106,858.

These and other process parameters, such as coating the liposomes or lipid/polymer liposomes with polymers are fully described in the art and can easily be applied to the manufacture of the compositions of this invention by any person having ordinary skill in the art. The liposomes and microparticles of the invention may be present in suspension for delivery. Useful suspending agents are substantially isotonic, for example, having an osmolarity of about 250-350 mOsM. Normal saline is particularly useful.

Methods of Administration

The resulting liposome-NS and microshere-NS preparations provide for the sustained release of the agents encapsulated therein. The compositions of the invention can be administered parenterally by injection or by gradual infusion over time. The compositions can be administered intravenously, intraperitoneally, intramuscularly, subcutaneously, intracavity, transdermally or via inhalation. The pharmaceutical compositions of the invention also can be administered enterally. Methods of administration include use of conventional (needle) and needle-free syringes, as well as metered dose inhalers (MDIs), nebulizers, spray bottles and intratracheal tubes.

Other methods of administration will be known to those skilled in the art. For some applications, such as subcutaneous administration, the dose required may be quite small, but for other applications, such as intraperitoneal administration, the required dose may be very large. While doses outside the foregoing dosage range may be given, this range encompasses the breadth of use for practically all physiologically active substances.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In case of conflict, the present specification, including definitions, will control. In addition, the materials, methods and examples are illustrative only and not intended to be limiting.

### EXAMPLE 1

### Preparation of Glibenclamide

Nanosuspension
Equipment

Ultra Turrax, IKA (Fischer AG, CH)
Kinematica PT 3100 (Kinematica, CH)
AVESTIN C5/C50, AVESTIN, (Canada)
COULTER LS230, COULTER (IG AG, CH)
MALVERN Zetasizer 3000 MS, GMP (CH)

Method per EP 605497 B

| | | |
|---|---|---|
| GLIBENCLAMIDE | KN 96089/1 | 20.0% W/W |
| Tween ® 80V | KN 99280/1 | 0.50% w/w |
| Plasdone ® K29-32 | KN 98131 | 0.50% w/w |
| Water for Injection | | 79.00% w/w |

Glibenclamide was supplied by FLARER SA (CH)
Plasdone ® K 29-32 was supplied by ISP AG (CH)
Tween ® 80 was supplied by QUIMASSO (F)

Preparation of an aqueous solution of Tween® 80V (120 ml): Tween® 80V and Plasdone® K29-32 were incorporated into water for injection under magnetic stirring until a clear solution was obtained. The slurry was then obtained by wetting glibenclamide with the appropriate quantity of the aqueous solution of surfactant. The resulting suspension was dispersed using a high shear, dispersing instrument (Ultra Turrax) for 1 minute at 11,000 rpm. The suspension was left for 30 min. under magnetic agitation (200 rpm) to eliminate foaming. The resulting parent suspension (150 ml) was passed through a high-pressure piston gap homogenizer (C50, continuous process and “cooling” system which resulted in a temperature around 20° C. (19°-21° C.)) to obtain a nanosuspension. The operational parameters were set up as follows: Homogenization pressure: 1500 bars

Processing time: 180 min.

Pre-homogenization step: 3 min. at 500 bars

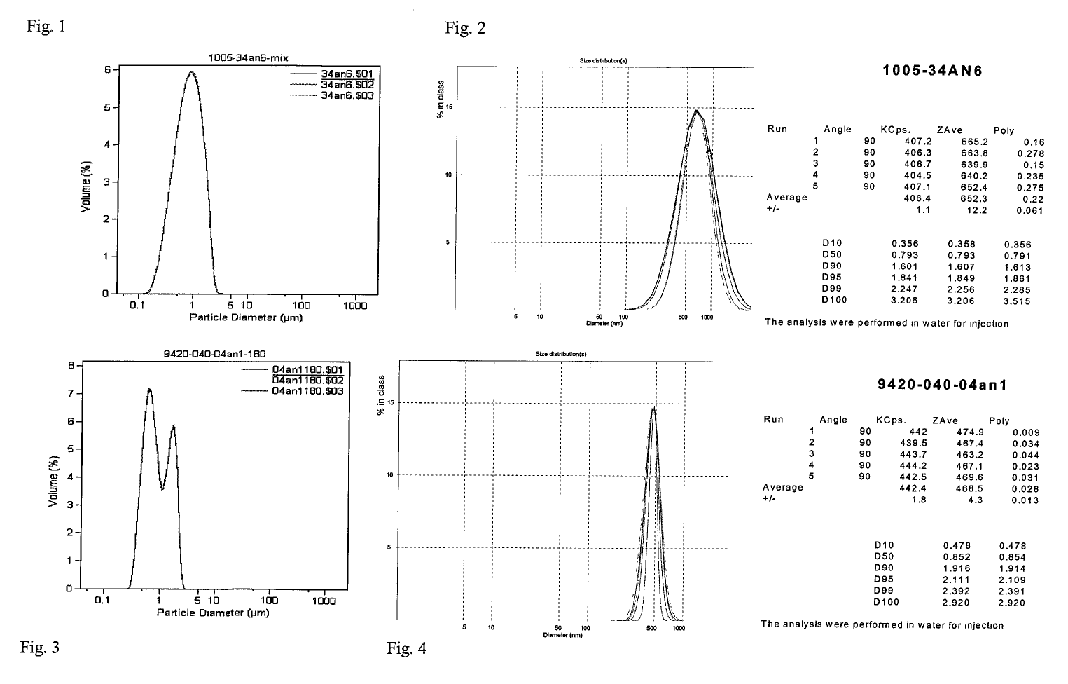

The particle sizes of the suspension and the resulting nanosuspension were measured using laser diffractometry (LD, Coulter LS 230) and by Photon Correlation Spectroscopy (Malvern, Zetasizer 3000MS) and the results are shown in FIGS. 1 and 2.

### EXAMPLE 2

### Preparation of Nifedipine

Nanosuspension
Equipment

Ultra Turrax, IKA (Fischer AG, CH)
Kinematica PT 3100 (Kinematica, CH)
AVESTIN C5/C50, AVESTIN, (Canada)
COULTER LS230, COULTER (IG AG, CH)
MALVERN Zetasizer 3000 MS, GMP (CH)

Method per EP 605497 B

| | | |
|---|---|---|
| Nifedipine | KN97081/1 | 10.0% w/w |
| Tween ® 20 | KN 99277/1 | 0.50% w/w |
| Plasdone ® K29-32 | KN 98131 | 0.50% w/w |
| Sodium dihydro- | | 89.00% w/w |
| genophosphate in water | | |
| for injection (10⁻²M) | | |

Nifedipine was supplied by FLARER SA (CH)
Plasdone ® K 29-32 was supplied by ISP AG (CH)
Tween ® 20 was supplied by QUIMASSO (F)
Sodium dihydrogenophosphate was supplied by MERCK (D)

Preparation of an aqueous solution of Tween® 20 and Plasdone® K29-32: Tween 20® and Plasdone® K 29-32 were incorporated into water for injection under magnetic stirring until a clear solution was obtained. The slurry was then obtained by wetting nifedipine with the appropriate quantity of the aqueous solution of surfactant. The resulting suspension was dispersed using a high shear dispersing instrument (KINEMATICA PT 3100) for 1 min. at 11,000 rpm. The suspension was left for 30 min. under magnetic agitation (200 rpm) to eliminate foaming. The resulting parent suspension (slurry, 40 ml) was passed through a high-pressure piston gap homogenizer (C5, continuous process and “cooling” system which resulted in a temperature around 14° C. (12° C.-16° C.) to obtain a nanosuspension. The operational parameters were set up as follows:

Homogenization pressure: 1500 bars
Processing time: 90 min
Pre-homogenization step: 4 cycles at 500 bars

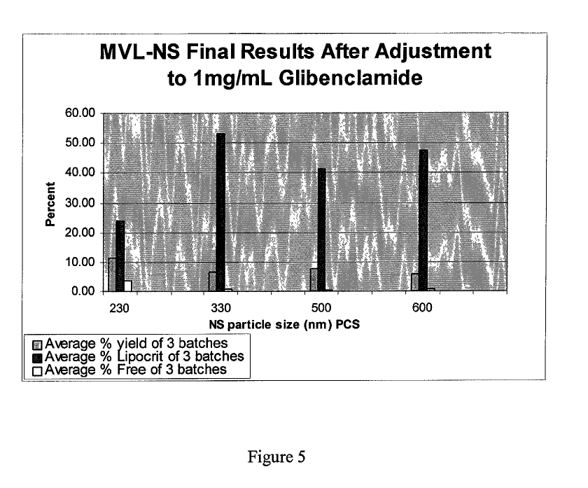

The particle sizes of the suspension and the resulting nanosuspension were measured using laser diffractometry (LD, Coulter LS 230) and by Photon Correlation Spectroscopy (Malvern, Zetasizer 3000MS) and the results are shown in FIGS. 4 and 5.

### EXAMPLE 3

### Preparation of Multivesicular Liposomes

Multivesicular liposome particles were prepared by a double emulsification process. All formulations were prepared using an organic solvent phase, consisting of the stated solvent with 1% ethanol, and a mixture of phospholipids, cholesterol, and triglycerides. Nanosuspensions containing glibenclamide were used as the first aqueous phase with the osmolarity adjusted with dextrose. The first aqueous phase was mixed with the solvent phase at high speed (9000 rpm for 8 minutes) on a TK Homo mixer, forming a water-in-oil emulsion. This emulsion was then mixed at low speed (4000 rpm for 1 minute) with the second aqueous phase (4% glucose monohydrate and 40 mM lysine), forming a water-in-oil-in-water emulsion. The solvent was evaporated and the particles were recovered and washed by centrifugation. The pellets were resuspended in 10 grams of saline unless otherwise specified. Generally, the steps to follow when performing a double emulsion process are as follows: First, a water-in-oil type emulsion is formed from a “first” aqueous phase and a volatile organic solvent phase. The first aqueous phase also may contain excipients such as osmotic spacers, acids, bases, buffers, nutrients, supplements or similar compounds. The first aqueous phase may contain a natural, synthetic or genetically engineered chemical or biological compound that is known in the art as having utility for modulating physiological processes in order to afford diagnosis of, prophylaxis against, or treatment of, an existing or pre-existing condition in a living being. The water-in-oil type emulsion can be produced by mechanical agitation such as by ultrasonic energy, nozzle atomization, by the use of static mixers, impeller mixers or vibratory-type mixers. Forcing the phases through a porous pipe to produce uniform sized emulsion particles also can form such emulsions. These methods result in the formation of solvent spherules. This process may be repeated using different starting materials to form multiple “first” aqueous phases such that a variety of types of solvent spherules are used in subsequent steps.

Second, the solvent spherules which are formed from the first water-in-oil type emulsion are introduced into a second aqueous phase and mixed, analogously as described for the first step. The second aqueous phase can be water, or may contain electrolytes, buffer salts, or other excipients well known in the art of semi-solid dosage forms, and preferably contains glucose and lysine. The “first” and “second” aqueous phases may be the same or different.

Then, the volatile organic solvent is removed, generally by evaporation, for instance, under reduced pressure or by passing a stream of gas over or through the spherules. Representative gases satisfactory for use in evaporating the solvent include nitrogen, helium, argon, carbon dioxide, air or combinations thereof. When the solvent is substantially or completely removed, the lipid-containing composition is formed with the desired agent encapsulated in biodegradable liposomes formed from the lipid components, with the liposomes suspended in the second aqueous phase. Lipid/polymer combinations also may be used to form the vesicle bi-layers.

If desired, the second aqueous phase may be exchanged for another aqueous phase by washing, centrifugation, filtration, or removed by freeze-drying or lyophilization to form a solid dosage. The solid dosage form of the pharmaceutical composition obtained, by, for example freeze-drying, may be further processed to produce tablets, capsules, wafers, patches, suppositories, sutures, implants or other solid dosage forms known to those skilled in the art.

### EXAMPLE 4

### Effects of NS Particle Size on MVL Encapsulation

Four bottles containing glibenclamide nanosuspension of different sizes arrived from SkyePharma AG Muttenz without any apparent aggregation. The bottles were designated as 9420-040-2527B, 9420-040-04AN, 9420-040-17An, and 9420-040-18AN. Each bottle contained glibenclamide nanoparticles of different sizes. The nanosuspensions were made with 20% glibenclamide (200 mg/mL), 0.5% polyvinyl pyrrolidone (PVP) and polyoxyethylene sorbitan monooleate (Tween® 80). The samples were assayed for pH and osmolarity; the results are in the following table.

| | Diameter (PCS, | | |
|---|---|---|---|
| Volume | Osmolarity | | |
| Samples | weighted nm) | (mmol/Kg) | pH |
| 9420-040- | 230 | 45 | 7.9 |
| 2527B | | | |
| 9420-040- | 330 | 50 | 9.4 |
| 04AN | | | |
| 9420-040- | 500 | 47 | 9.6 |
| 17AN | | | |
| 9420-040- | 600 | 50 | 9.7 |
| 18AN | | | |

MVL batches were made using these four nanosuspensions as a first aqueous phase. The osmolarity was adjusted with dextrose, and the lipid combination (triolein 2.4 mM, cholesterol 19.9 mM, DOPC 13.2 mM, and DOPG, sodium salt 2.8 mM) was dissolved in isopropyl ether with 1% ethanol. The mixing conditions were 9000 rpm for 8 minutes for the first emulsion, 4000 rpm for 1 minute for the second emulsion, and gentle rotary shaking at 37° C. while being flushed with nitrogen for 40-60 minutes to remove solvent. When MVL batches were made using undiluted glibenclamide nanosuspension, no MVL particles were recovered.

A second set of batches was made with the nanosuspension diluted 10-fold, containing 2% glibenclamide and 0.05% each PVP and Tween® 80, and the osmolarity adjusted to about 290 mmol/Kg with dextrose. The batches were assayed by HPLC to determine percent encapsulation and percent of unencapsulated (free) drug. Because the drug is particulate, it is probable that some unencapsulated drug is found in the pellet fraction. If so, the percent free drug, which is operationally defined as the proportion of drug found in the supernatant, may be underestimated. In the following results, MVL suspensions were adjusted to 1 mg/mL of glibenclamide. The results are in the tables below and in FIG. 5.

MVL particle characterization includes determination of percent yield, packed particle volume (lipocrit), percent free drug, drug loading, percent drug loading, and particle size distribution. These assays are defined as follows: Percent yield of drug is the percentage of drug used in producing the formulation that is recovered in the final product. Lipocrit is the ratio of the pellet volume to the suspension volume. Percent free drug is the amount of drug that is in the supernatant, expressed as a percentage of the total amount of drug in the suspension. The drug loading is defined as the concentration of drug in the particle fraction of the suspension. It is expressed as mg of drug per mL of packed particles. The percent loading is a ratio of the drug loading concentration to the drug concentration in the first aqueous phase used to make the particles. Particle size distribution and the mean diameter are determined by the method of laser light scattering using an LA-910 Particle Analyzer from Horiba Laboratory Products, Irvine, Calif.

| Nanoparticle | | | | Mean Diameter |
|---|---|---|---|---|
| Diameter by | Yield | Lipocrit | Free | Volume Weighted |
| PCS (nm) | (%, ± s.d.) | (%, ± s.d.) | (%, ± s.d.) | (μm, ± s.d.) |
| 230 | 11.3 ± 0.2 | 23.8 ± 4.3 | 3.7 ± 5.9 | 26.4 ± 3.5 |
| 330 | 6.7 ± 2.0 | 52.9 ± 19.8 | 0.9 ± 0.6 | 21.5 ± 1.7 |
| 500 | 7.6 ± 2.7 | 41.1 ± 9.3 | 0.3 ± 0.2 | 25.7 ± 2.8 |
| 600 | 5.9 ± 1.6 | 47.5 ± 16.6 | 0.9 ± 1.1 | 21.1 ± 2.7 |

These results show that MVL-encapsulated glibenclamide nanosuspensions can be made reproducibly. It was expected that the yield would increase with a decrease in particle size. Although no clear correlation was established, it appears that the highest yield was achieved with nanoparticles 230 nm in size.

To establish a clearer trend in the effects of particle size on yield, and to determine if it is possible to increase the yields by decreasing the drug concentration, MVL batches were made using glibenclamide nanosuspensions diluted 10, 50 and 100-fold.

It should be noted that when the following MVL batches were made, the nanosuspensions had settled out of solution. The nanoparticles could be resuspended by gentle shaking. Any particle size changes could not be confirmed with a laser scattering particle size distribution analyzer.

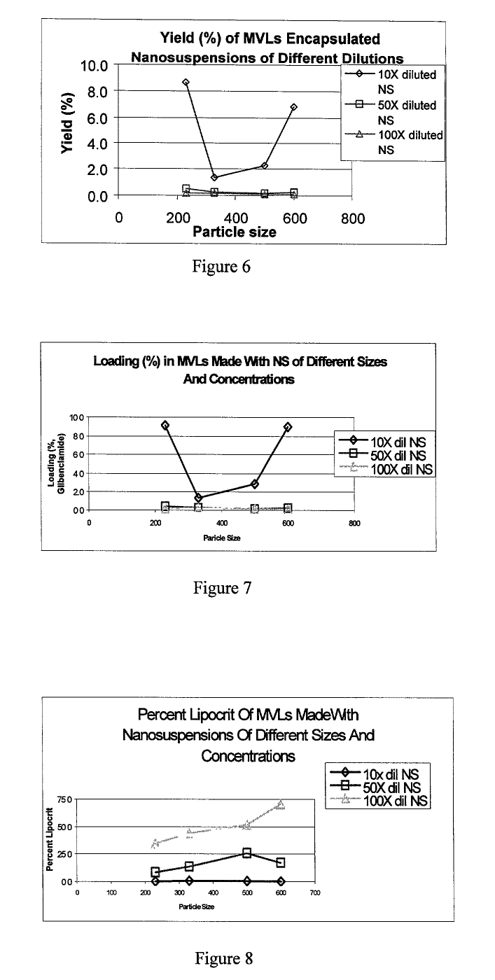

Three sets of batches were made with the nanosuspensions diluted 10-fold (2% glibenclamide, and 0.05% each PVP, and Tween® 80), 50-fold (0.4% glibenclamide and 0.01% each PVP and Tween® 80), and 100-fold (0.02% glibenclamide and 0.005% each PVP and Tween® 80). The osmolarity was adjusted to about 290 mmol/Kg with dextrose. The batches were assayed by HPLC to determine percent encapsulation and percent of unencapsulated drug in the supernatant. The concentrations of the MVL particles made with nanosuspension diluted 100-fold were adjusted to 2 μg/mL of glibenclamide. The MVLs made with nanosuspensions diluted 10- and 50-fold could not be adjusted to 2 μg/mL and have a measurable lipocrit; therefore, the lipocrit values shown here for the 10- and 50-fold MVL batches are the extrapolated values if it were diluted to that concentration. The results are in the table below and in FIGS. 6-8.

| | | | | | | | Diameter |
|---|---|---|---|---|---|---|---|
| NS | | | | | | Load- | Volume |
| Particle | | Yield | Lipocrit | Free | Loading | ing | Weighted |
| Size | Dilution | (%) | (%) | (%) | (mg/mL) | (%) | (μm) |
| 230 | 10x | 8.6 | 0.1 | 1.5 | 1.8 | 9.1 | 24.6 |
| 330 | 10x | 1 3 | 0 7 | 9 1 | 0 2 | 1 4 | 24 6 |
| 500 | 10x | 2.3 | 0.5 | 0.9 | 0.4 | 2.9 | 24.6 |
| 600 | 10x | 6.8 | 0.1 | 0.5 | 1.6 | 9.0 | 24.6 |
| 230 | 50x | 0.5 | 8.4 | 32.1 | 0.0 | 0.4 | 22.0 |
| 330 | 50x | 0.3 | 13.4 | 31.4 | 0.0 | 0.3 | 23.4 |
| 500 | 50x | 0.2 | 25.8 | 16.5 | 0.0 | 0.2 | 22.2 |
| 600 | 50x | 0.2 | 17.1 | 14.8 | 0.0 | 0.2 | 22.4 |
| 230 | 100x | 0.1 | 33.9 | 24.5 | 0.0 | 0.2 | 29.7 |
| 330 | 100x | 0.1 | 43.8 | 17.4 | 0.0 | 0.2 | 26.5 |
| 500 | 100x | 0.1 | 51.9 | 10.7 | 0.0 | 0.2 | 26.2 |
| 600 | 100x | 0.1 | 70.6 | 7.7 | 0.0 | 0.1 | 25.2 |

These results confirm previous findings for the 10-fold diluted glibenclamide nanosuspension that no clear correlation was established between yield of encapsulation and nanosuspension particle size. The hightest yield of encapsulation was achieved with n nanoparticles 230 nm in size.

### EXAMPLE 5

### Effects of PVP and Tween® on MVL Particles

MVL batches were made with polyoxyethylenesorbitan monooleate (Tween® 80) and polyvinyl pyrrolidone (PVP) in the first aqueous phase. This series of formulations did no contain glibenclamide. The osmolarity was adjusted with dextrose, and the lipid combination (triolein, cholesterol, DOPC, and DOPG) was dissolved in isopropyl ether with 1% ethanol. The mixing conditions were 9000 rpm for 8 minutes for the first emulsion, 4000 rpm for 1 minute for the second emulsion, and gentle rotary shaking at 37° C. with nitrogen for 40 minutes to remove solvent.

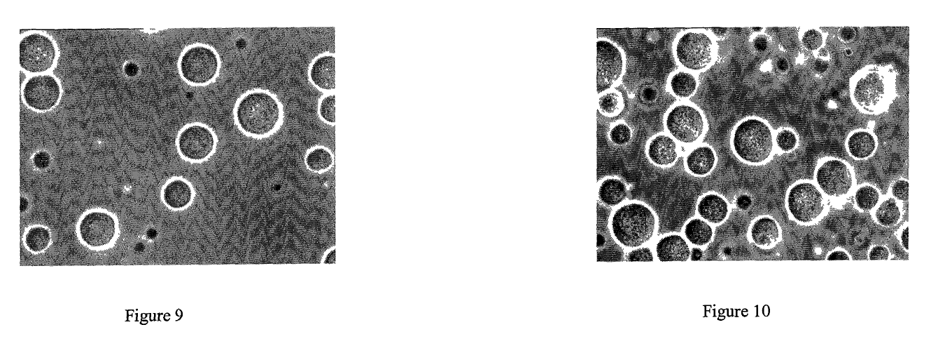

MVL particles were made using first aqueous phases containing 5% anhydrous dextrose and different concentrations, 0.5, 0.05, 0.005, and 0.005%, of PVP and Tween® 80. Particles were recovered for all batches. The micrographs representative of the particles recovered are seen in FIGS. 9 and 10.

The following are particle sizes and lipocrits of the batches made with concentrations of PVP and Tween® 80 varied in parallel.

| PVP and Tween 80 | Volume Weighted | Lipocrit |
|---|---|---|
| Concentration (%) | diameter (μm) | (%) |
| 0 | 22.2 | 47.4 |
| 0.0005 | 22.2 | 49.5 |
| 0.005 | 21.2 | 41.9 |
| 0.05 | 20.7 | 32.2 |
| 0.5 | 17.6 | 25.3 |

These results show that with increasing concentration of both PVP and Tween® 80 together, lipocrit and particle size decrease. Since the lipocrit is a reflection of the volume of first aqueous phase encapsulated, batches made with 0.5% Tween® 80 and 0.5% PVP encapsulate roughly half the volume of batches made without these ingredients.

In separate experiments, MVL batches were made to test the effects of PVP or Tween® varied individually. One set of batches contained 0.5% Tween® 80 kept constant, with PVP varying from 0.005 to 0.5%. In the second set of batches, the PVP was kept at 0.5% and the Tween® concentration was varied from 0.0005 to 0.5%. The following graphs and tables show the results of these two experiments.

MVLs made with first aqueous phase containing 0.5% Tween® and varying concentration of PVP:

| PVP Concentration | Volume Weighted | Lipocrit |
|---|---|---|
| (%) | Diameter (μm) | (%) |
| 0.0005 | 16.8 | 12.7 |
| 0.005 | 13.8 | 16.2 |
| 0.05 | 18.0 | 15.4 |
| 0.5 | 15.2 | 21.1 |

MVLs made with first aqueous phase containing 0.5% PVP and varying concentration of Tween®:

| Tween Concentration | Volume Weighted | Lipocrit |
|---|---|---|
| (%) | Diameter (μm) | (%) |
| 0.0005 | 20.7 | 46.7 |
| 0.005 | 23.0 | 44.6 |
| 0.05 | 18.0 | 28.7 |
| 0.5 | 15.2 | 21.1 |

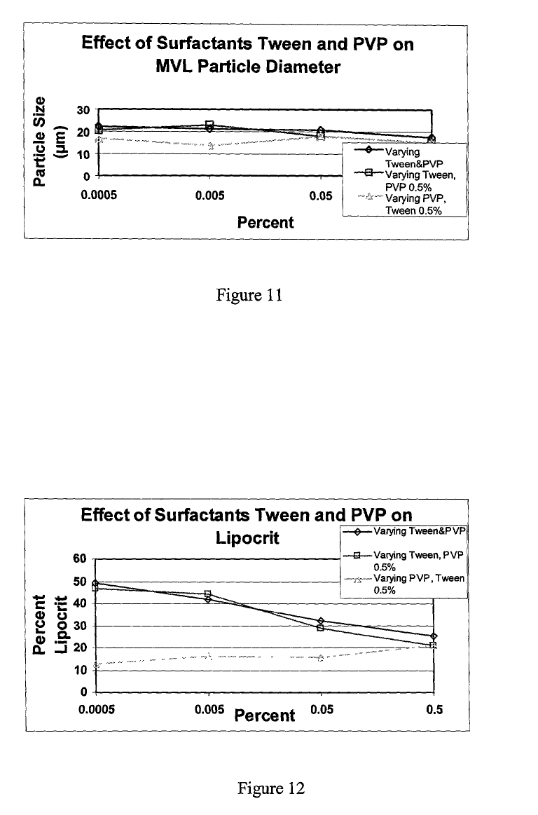

Further results are illustrated in FIGS. 11 and 12.

These results show that the presence of Tween® 80 in concentrations higher than 0.005% causes a slight decrease in particle diameter. However, the lipocrit of particles containing Tween® 80 decreases by as much as 50 percent. PVP has little effect on diameter or lipocrit, at least in the presence of 0.5% Tween® 80. In contrast, increasing the concentration of Tween® 80 has a clear deleterious effect on the lipocrit. This may explain the poor yield and low lipocrit seen with 10 fold-diluted nanosuspensions.

### EXAMPLE 6

### Effects of Different Solvents on Yield of MVL-Encapsulated Agent Nanosuspension 9420-040-04AN7

A glibenclamide nanosuspension were obtained from SkyePharma AG Muttenz. The bottles were all the same batch designated 9420-040-04AN7. The nanosuspension contained particles of 550 μm in diameter (measured by laser light diffraction using a Coulter® particle analyzer), 10% glibenclamide (100 mg/mL), and 0.5% each polyvinyl pyrrolidone (PVP) and polyoxyethylene sorbitan monooleate (Tween® 80). The formulation development was continued using this nanosuspension.

It was previously established that the lipid combination for making MVL-encapsulated nanosuspension particles could be dissolved in either isopropyl ether, pentane, 1,1,1-trichloroethane, or 1,1-dichloro-2-fluoroethane (Forane® 141b). To determine if there was an effect on yield with any one of these solvents, and to attempt to find a more practical solvent than isopropyl ether, MVL batches were made using all four solvents.

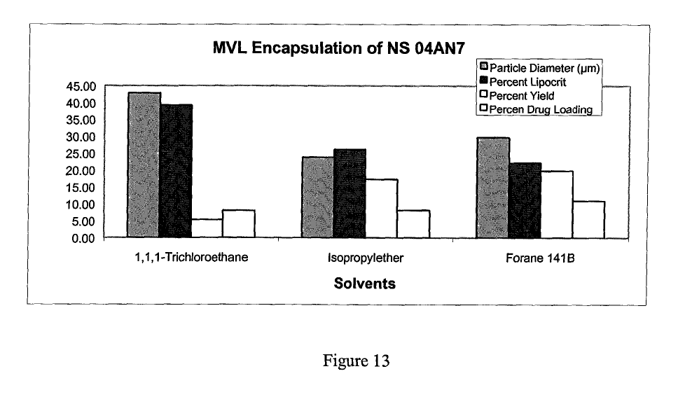

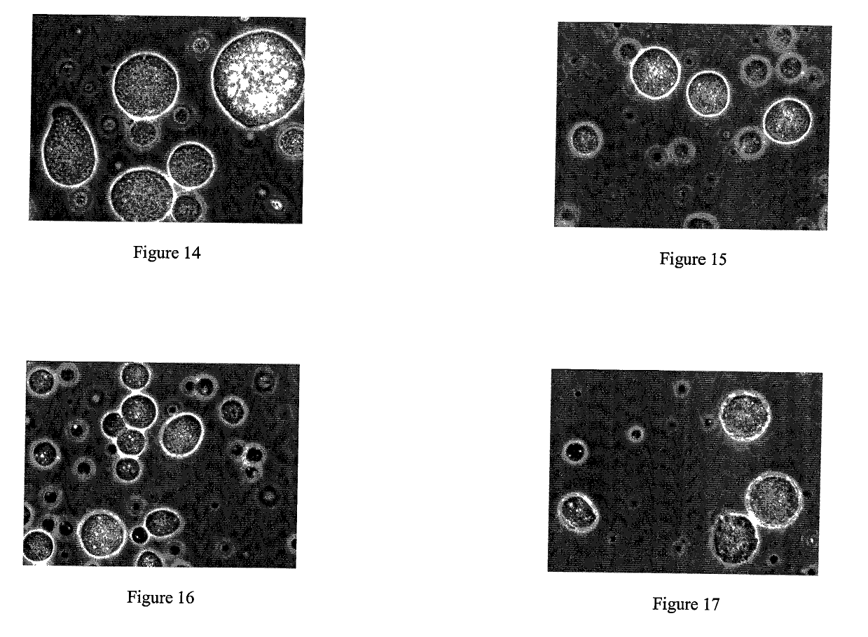

The results show that Forane® 141b is a good substitute for isopropyl ether. No MVL particles were recovered with pentane as a lipid solvent. Using 1,1,1-trichloroethane as the lipid solvent gave a low percent yield. The percent loading and percent yield of MVL-encapsulated glibenclamide nanosuspension is slightly higher with Forane 141b, 10% and 19% respectively, than with isopropyl ether, 8% and 17% respectively. The length-weighted particle size is similar with both solvents. Following is a table showing the results for these batches. Micrographs of the particles are illustrated in FIGS. 13-17.

| | | | | | | | Volume | Length |
|---|---|---|---|---|---|---|---|---|
| | | Lipocrit | Yield | Free | | Loading | Weighted | Weighted |
| Lipid Solvent | mg/mL | (%) | (%) | (%) | Loading | (%) | (μm) | (μm) |
| 1,1,1-Trichloroethane | 0.5 | 39.3 | 5.3 | 1.3 | 0.9 | 8.1 | 42.9 | 24.8 |
| Isopropyl ether | 0.5 | 26.1 | 17.3 | 0.5 | 0.9 | 8.2 | 23.8 | 19.5 |
| Forane 141B | 0.5 | 22.2 | 19.9 | 0.4 | 1.2 | 11.0 | 29.7 | 22.9 |

### EXAMPLE 7

### Morphology of MVL-Encapsulated Nanosuspensions

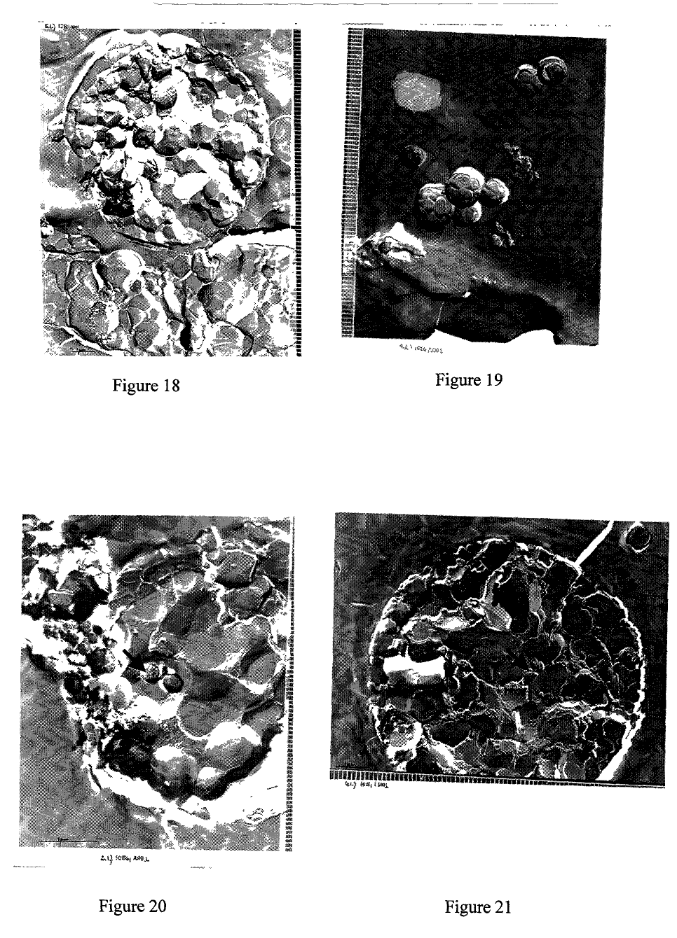

Electron micrographs (EM) of MVL-encapsulated nanosuspensions were performed by Dr. Papahadjopoulos-Sternberg, NanoAnalytical Laboratory, San Francisco. Nine samples were sent for freeze fracture electron microscopy including unencapsulated and MVL-encapsulated nanosuspensions (nanosuspension lot numbers: 2527B, 04AN, 17AN, and 18AN) and a MVL blank without any encapsulated nanoparticles. The purpose of sending these samples was to measure the nanosuspension particles before and after encapsulation and to visualize how the nanoparticles are encapsulated in the MVLs. The results are represented in FIGS. 18-21.

FIG. 18—MVL without nanoparticles (Blank) This micrograph of a blank MVL is a good representation of the internal chambers in MVL particles. The internal chambers can be measured to be between 1 and 3 μm in size and are well-defined with distinct facets.

FIG. 19—Nanosuspension 18AN

This lot of nanosuspension was assayed by Photon Correlation Spectroscopy (PCS) and has an average size of 600 nm, ranging between 150 nm-6 μm. The particles in this micrograph range in size between 250 and 500 nm. Because of their smooth spherical shape, they resemble a single internal chamber excised from a MVL particle.

FIG. 20—MVL-NS (04AN)

The nanoparticles in this suspension were measured by PCS to be an average of 330 nm with a range between 300-800 nm. This micrograph shows two small particles, approximately 300-400 nm, within an internal chamber of a MVL particle (noted by arrow). Nanoparticles also can be seen on the outside edge of the MVL.

FIG. 21—MVL-NS (18AN)

These particles were measured by Photon Correlation Spectroscopy (PCS) and have an average size of 600 nm, ranging between 150 nm-6 μm. This micrograph shows two small nanoparticles in the outer edges of internal chambers of a MVL particle (noted by arrow). They are approximately 400 nm in size.

Results:

The combined results of these studies show that:

Effects of Nanosuspension Particle Size on MVL Encapsulation

The highest yield of encapsulation was obtained with the nanosuspension containing 230 nm size particles.

There is a decrease in percent yield and drug loading when the nanosuspension is diluted 50- and 100-fold.

This suggests that unencapsulated drug is being measured in the pellet since aggregation and pelleting of unencapsulated nanoparticles as well as adsorption to the external surface of MVL particles, is more likely at higher concentration.

Effects of PVP and Tween® on MVL Particles

Tween® causes a difference in MVL particles.

Specifically, the presence of Tween® in concentrations higher than 0.005% causes a decrease in MVL particle size and lipocrit, even in the absence of nanoparticles.

Effects of Different Solvents on Yield of MVL-Encapsulated Drug

Forane® 141b is a good substitute for isopropyl ether as a lipid solvent. In one experiment, Forane® 141B gave 15% better yield.

Morphology of MVL Encapsulated Nanosuspensions

Nanoparticles were encapsulated into MVL.

Considering the spherical appearance and size of the nanosuspensions in FIG. 19, only the smallest nanoparticles can be clearly identified in the interior of MVL.

Micrographs show that the nanoparticles can be found associated with MVL on the outside as well as encapsulated in the internal chambers.

### EXAMPLE 9

### Bioavailability of MVL-Encapsulated Perphenazine Solution and Perphenazine Nanosuspension

In this study perphenazine was prepared as a nanosuspension by mechanical means. Bioavailability of perphenazine nanosuspension and MVL encapsulated perphenazine solution were examined in rats upon subcutaneous administration. Perphenazine was present in rat serum for 30 days for MVL encapsulated perphenazine solution. Serum concentrations were detectable for up to 2 days for perphenazine nanosuspension and 24 hr for perphenazine solution. Controlled release of perphenazine nanosuspension from MVL particles was examined in vitro at 37° C. in human plasma.

Poorly soluble drugs can be solubilized by reducing the size of drug particles (300 to 800 nm in diameter) in the presence of surfactants. An increase in the dissolution rate would be possible by further increasing the surface of the drug powder. Perphenazine, an antipsychotic drug, is highly insoluble in water. To increase the bioavailability of the drug, perphenazine nanosuspension was made. Nanosuspensions were encapsulated into the aqueous chambers of MVL particles, so that insoluble perphenazine could be delivered via parenteral routes with the benefit of sustained release. At acidic pH, perphenazine is soluble in aqueous medium. Throughout this example, “perphenazine solution” refers to the perphenazine solubilized in 15 mM sodium citrate buffer (pH 4.0).

Materials: DOPC (1,2-dioleoyl-sn-glycero-3-phosphocholine), DOPG (1,2-dioleoyl-sn-glycero-3-phosphoglycerol), and triolein (1,2,3-trioleoylglycerol) were from Avanti Polar Lipids Inc. (Alabaster, Ala.). Cholesterol and chloroform were from Spectrum Chemical Manufacturing Corporation (Gardena, Calif.). Perphenazine was from Sigma Chemical Co. (St. Louis, Mo.).

Perphenazine nanosuspension: Perphenazine was homogenized at a concentration of 10 mg/mL in a solution containing 7.5% (w/v) sucrose, 10 mM phosphate buffer, pH 7.3, 15 mM Glycine, and 0.05% (w/v) Tween® 20. (261 mOsm) using a Polytron mixer (Brinkman, PT3000). The solution was kept on ice while mixing. Perphenazine solution was mixed for 10 cycles at 20,000 rpm (30 sec. on, 30 sec. off to control temperature); 30 cycles at 25,000 rpm (30 sec. on, 30 sec. off); 10 cycles at 25,000 rpm (2 minutes on, 1 minute off).

This solution was processed through an extruder (Northern Lipids) at 100-300 lbs. of pressure. The solution was extruded sequentially through 5.0 μm, 1.0 μm, 0.3 μm and 0.1 μm polycarbonate filters. The mean particle size of the resulting suspension was determined using a laser scattering particle size distribution analyzer (Horiba LA-910, Horiba Instruments, Irvine, Calif.). Perphenazine concentration was measured on HPLC using a reverse phase C18 column (Primesphere 250×4.6 mm, 5 μm, Phenomenex) using a mobile phase comprised of 38% 50 mM acetate pH 4, 52% ACN, 10% MeOH. Perphenazine was detected at a wavelength of 257 nm.

MVL encapsulated perphenazine nanosuspension: 5 mL of perphenazine nanosuspension was combined with 5 mL of solvent phase containing 2.2 g/L Triolein, 7.7 g/L cholesterol, 10.4 g/L DOPC and 2.22 g/L DOPG in forane (CC12FCH₂). Perphenazine nanosuspension was added 1 mL at a time and mixed at 9000 rpm in a TK mixer for 8 min. Further 20 mL of glucose/lysine solution (45 mL water, 1 mL of 2M lysine and 4 mL of 50% (w/v) glucose) was added and dispersed at 4000 rpm for 1 minute. MVL were formed by removing solvent at 37° C. by flushing N₂ over the solution for 60 minutes. 20 mL of water was added at 20 minute and 40 minute time intervals. Particles were recovered by centrifuging at 3000 rpm for 10 min in PBS (450 mL saline, 50 mL 10 mM phosphate buffer, pH 8.0) solution. Particles were resuspended in the same solution as 50% (w/v) suspension. Perphenazine concentration in MVL particles was measured using HPLC as described earlier.

MVL encapsulated perphenazine solution: The aqueous phase contained perphenazine (2 mg/mL) in 15 mM sodium citrate buffer (pH 4.0). At acidic pH perphenazine is soluble in the citrate buffer. Equal amounts (5 mL) of an aqueous phase and a solvent phase were mixed at high speed (9,000 rpm for 8 minutes followed by 4,000 rpm for 1 minute) on a TK mixer to form a water-in-oil emulsion. The solvent phase contained 10.4 mg/mL DOPC, 2.1 mg/mL DPPG, 7.7 mg/mL cholesterol, and 2.2 mg/mL triolein dissolved in chloroform. Twenty milliliters of an aqueous solution containing glucose (32 mg/mL) and lysine (40 mM) were added to the emulsion and stirred (4,000 rpm for 1 min) to disperse the water-in-oil emulsion into solvent spherules. MVL were formed by removing chloroform at 37° C. by flushing N₂ over the solution (50 L/min). Solvent was removed from suspensions in a water bath at 100 rpm for 20 minutes. The MVL particles were recovered by centrifugation at 600× g for 10 min and washed twice in saline (0.9% NaCl). MVL particles were resuspended in saline as 50% suspensions (w/v). The mean particle diameter was determined on a laser-scattering particle size distribution analyzer. Particles were observed under the light microscope for morphological appearance. Perphenazine content in the MVL formulations was measured on a reverse phase C18 column with following dimensions: 4.6×250 mm, 5 μm (Primesphere, Phenomenex) using mobile phase (52% acetonitrile, 10% methanol, 38% acetate buffer at pH 4.0).

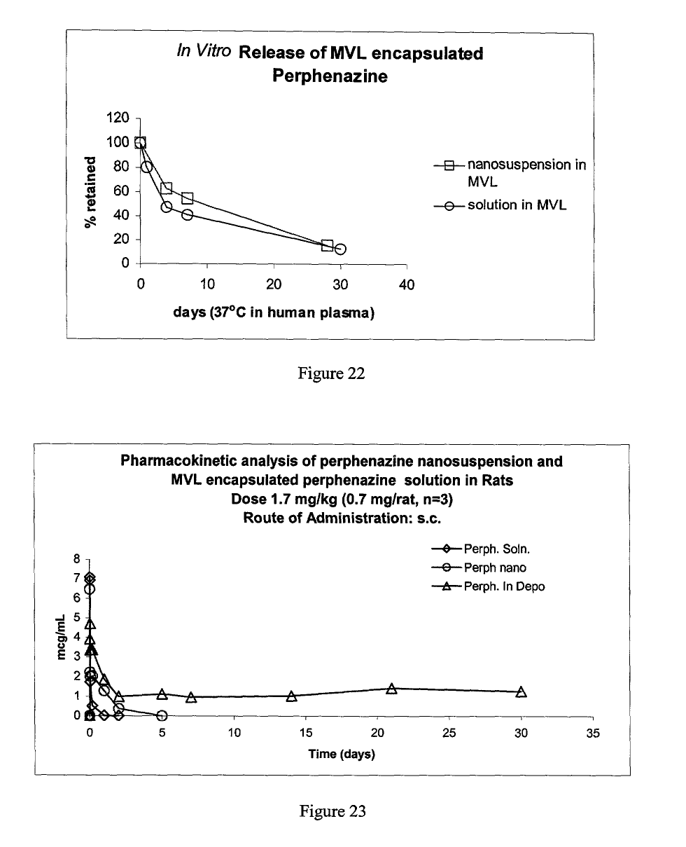

In Vitro Release Assay: The MVL particle suspensions were diluted in human plasma to achieve a final 10% (w/v) suspension. The MVL particle suspension (0.5 mL) was diluted with 1.2 mL of human plasma with 0.01% sodium azide (Sigma, St. Louis, Mo.) in screw-cap 2 mL polypropylene tubes (Eppendorf) and placed at 37° C. under static conditions. Samples were taken for analyses according to the planned schedule after measuring pellet volume in each sample, particle pellets were harvested by centrifugation in a micro-centrifuge at 16,000× g for 4 min. and stored frozen at −20° C. until assayed. Perphenazine content in pellets was extracted with mobile phase (52% acetonitrile, 10% methanol, 38% acetate buffer at pH 4.0) and analyzed on HPLC using a C18 column as described above. The results are shown in FIG. 22.

In Vivo experiments and sample analysis: Perphenazine solution, perphenazine nanosuspension, and MVL encapsulated perphenazine solution were injected subcutaneously at a dose of 0.7 mg in 1 mL volume in male Sprague-Dawley rats (Harlan Sprague Dawley). Rats weighed approximately 350 g at study initiation. Serum samples (100 μL) were collected at 15 min., 30 min., 1 hr., 4 hr., 24 hr., 48 hr., 5 day, 7 day, 14 day, 21 day and 30 day time points.

Each 100 μL serum sample was added to 480 μL of ethyl acetate/hexane (2:1) solution and 8 μL of 1M NaOH. After vigorous mixing for 30 s, the samples were centrifuged at 2000 rpm for 3 min. 360 μL of organic phase was removed to a separate vial. This extraction step was repeated and to a pooled 720 μL of organic phase, 200 μL of 0.1M HCl were added. The samples were mixed and centrifuged as before. The organic phase was discarded and 8 μL of 6M NaOH and 240 μL of hexane were added to the aqueous phase. The samples were mixed and centrifuged. An aliquot of 200 μL of organic phase was collected. After evaporating the organic solvents under nitrogen, 75 uL of mobile phase (38% 50 mM acetate at pH 4.0, 52% ACN, 10% MeOH) were added to each HPLC vial and the samples were analyzed for perphenazine content on a C18 reverse phase column (5 μm, 250×4.6 mm).

Results: Perphenazine nanosuspensions were prepared by mechanical homogenization followed by extrusion through a gradient of polycarbonate filters under pressure. The mean particle size of the resulting suspension was determined as ˜380 nm using a laser scattering particle size distribution analyzer. Perphenazine nanosuspension was encapsulated into the aqueous chambers of MVL particles as described in the methods.

Rate of release of the encapsulated perphenazine both in solution and in nanosuspension forms into human plasma was determined for MVL particles using an in vitro assay. Time points were set up using 2 mL polypropylene tubes containing 1.2 mL of human plasma with 0.01% sodium azide and 0.5 mL sample suspension and placed at 37° C. under static conditions. The percentage of perphenazine retained by the MVL particles as a function of time relative to that at time zero indicates a sustained release of the encapsulated perphenazine over a 30-day period (FIG. 22). In both perphenazine solution and nanosuspension containing MVL particles, the rate of release is comparable.

A comparative evaluation of perphenazine serum concentrations over time for perphenazine nanosuspension and MVL encapsulated perphenazine solution was carried out in Harlan Sprague Dawley normal male rats. Doses (0.7 mg) were injected subcutaneously into the right lateral hind limb. For each study, three rats were used. The injection volume was kept constant at 1 mL.

A detectable level of perphenazine was present in rat serum for 30 days when MVL encapsulated perphenazine solution was administered. When a similar dose of perphenazine was administered as nanosuspension, serum concentrations were detectable for up to 2 days. Serum concentrations peaked and returned to basal level within 24 hr when same does of perphenazine solution was administered (FIG. 23).

The following table shows the pharmacokinetic parameters of perphenazine in rats:

| | | | perphenazine |
|---|---|---|---|
| | Perphenazine | Perphenazine | solution in |
| | solution | nanosuspension | DepoFoam |
| Cₘₐₓ | 7.08 | 6.75 | 4.70 |
| Tₘₐₓ | 15 | 15 | 30 |
| AUC | 0.570729 | 3.108906 | 37.10438 |

At a given dose, Cₘₐₓ for MVL encapsulated perphenazine is lower than the Cₘₐₓ for perphenazine solution. MVL encapsulated perphenazine solution exhibits characteristics of sustained release drug delivery (i.e., reduction in Cₘₐₓ and increase in mean resident time). Rat behavioral changes upon dose administration are well coincided with these results. Perphenazine is an antipsychotic drug and functions as a sedative. Rats administered with perphenazine solution are completely immobilized, where as the same doses of perphenazine nanosuspension or MVL encapsulated perphenazine solution did not show any noticeable changes in the animal behavior.

What is claimed is:

1. A liposome comprising at least one hydrophobic agent dispersed in at least one chamber bounded by at least one membrane.

2. A liposome as in claim 1, wherein said at least one hydrophobic agent is a nanoparticle.

3. A liposome as in claim 2, wherein said nanoparticle is in a nanosuspension.

4. A liposome as in claim 2, wherein said nanoparticle has size ranging from about 1 nm to about 1 micron.

5. A multivesicular liposome comprising at least one hydrophobic agent dispersed in at least one chamber bounded by at least one membrane.

6. A multivesicular liposome as in claim 5, wherein said at least one hydrophobic agent is a nanoparticle.

7. A multivesicular liposome as in claim 6, wherein said nanoparticle is in a nanosuspension.

8. A multivesicular liposome as in claim 6, wherein said nanoparticle has size ranging from about 1 nm to about 1 micron.

9. A microsphere comprising at least one hydrophobic agent dispersed in at least one internal chamber bounded by at least one membrane.

10. A microsphere as in claim 9, wherein said at least one hydrophobic agent is a nanoparticle.

11. A microsphere as in claim 10, wherein said nanoparticle is in a nanosuspension.

12. A microsphere as in claim 10, wherein said nanoparticle has size ranging from about 1 nm to about 1 micron.

13. A liposome as in claim 1, wherein said at least one hydrophobic agent is further present in said at least one membrane.

14. A multivesicular liposome as in claim 5, wherein said at least one hydrophobic agent is further present in said at least one membrane.

15. A liposome as in claim 1, wherein said at least one membrane is formed by at least one lipid and at least one polymer in at least one bi-layer.

16. A multivesicular liposome as in claim 5, wherein said at least one membrane is formed by at least one lipid and at least one polymer in at least one bi-layer.

17. A mutivesicular liposome as in claim 5, wherein multiple hydrophobic agents are present in the same of at least one chamber.

18. A multivesicular liposome as in claim 17, wherein at said multiple hydrophobic agents are nanoparticles.

19. A multivesicular liposome as in claim 18, wherein said nanoparticles are in at least one nosuspension.

20. A multivesicular liposome as in claim 18, wherein said nanoparticles have size ranging from about 1 nm to about 1 micron.

21. A multivesicular liposome as in claim 19, wherein said multiple hydrophobic agents are nanoparticles in a single nanosuspension.

22. A multivesicular liposome as in claim 21, wherein said nanoparticles have size ranging from about 1 nm to about 1 micron.

23. A mutivesicular liposome as in claim 5, wherein multiple hydrophobic agents are present in at least two different said chambers.

24. The multivesicular liposome as in claim 23, wherein said multiple hydrophobic agents are nanoparticles.

25. The multivesicular liposome as in claim 24, wherein said nanoparticles are in nanosuspensions.

26. The multivesicular liposome as in claim 24, wherein said nanoparticles have size ranging from about 1 nm to about 1 micron.

27. A composition comprising at least one liposome comprising at least one hydrophobic agent dispersed in at least one chamber bounded by at least one membrane, and a pharmaceutically acceptable suspending agent.

28. A composition as in claim 27, wherein said at least one hydrophobic agent is a nanoparticle.

29. A composition as in claim 28, wherein said nanoparticle is in a nanosuspension.

30. A composition as in claim 28, wherein said at least one hydrophobic agent has size ranging from about 1 nm to about 1 micron.

31. A composition as in claim 28, wherein said at least one hydrophobic agent is perphenazine and said pharmaceutically acceptable suspending agent is substantially isotonic.

32. A composition comprising at least one multivesicular liposome comprising at least one hydrophobic agent dispersed in at least one chamber bounded by at least one membrane, and a pharmaceutically acceptable suspending agent.

33. A composition as in claim 32, wherein said at least one hydrophobic agent is a nanoparticle.

34. A composition as in claim 33, wherein said nanoparticle is in a nanosuspension.

35. A composition as in claim 33, wherein said at least one hydrophobic agent has size ranging from about 1 nm to about 1 micron.

36. A composition as in claim 33, wherein said at least one hydrophobic agent is perphenazine and said pharmaceutically acceptable suspending agent is substantially isotonic.

37. A composition comprising at least one microsphere comprising at least one hydrophobic agent dispersed in at least one internal chamber bounded by at least one membrane.

38. A composition as in claim 37, wherein said at least one hydrophobic agent is a nanoparticle.

39. A composition as in claim 38, wherein said nanoparticle is in a nanosuspension.

40. A composition as in claim 38, wherein said at least one hydrophobic agent has size ranging from about 1 nm to about 1 micron.

41. A composition as in claim 38, wherein said at least one hydrophobic agent is perphenazine and said pharmaceutically acceptable suspending agent is substantially isotonic.

42. A method for the sustained release of at least one hydrophic agent to a living being comprising administration to said living being of at least one liposome comprising the at least one hydrophic agent located within at least one liposome chamber.

43. A method as in claim 42, wherein said at least on hydrophobic agent is a nanoparticle.

44. A method as in claim 43, wherein said nanoparticle is in a nanosuspension.

45. A method as in claim 43, wherein said at least one hydrophobic agent has size ranging from about 1 nm to about 1 micron.

46. A method for the sustained release of at least one hydrophic agent to a living being comprising administration to said living being of at least one multivesicular liposome comprising the at least one hydrophic agent located within at least one multivesicular liposome chamber.

47. A method as in claim 46, wherein said at least on hydrophobic agent is a nanoparticle.

48. A method as in claim 47, wherein said nanoparticle is in a nanosuspension.

49. A method as in claim 47, wherein said at least one hydrophobic agent has size ranging from about 1 nm to about 1 micron.

50. A method for the sustained release of at least one hydrophic agent to a living being comprising administration to said living being of at least one microsphere comprising the at least one hydrophic agent located within at least one microsphere chamber.

51. A method as in claim 50, wherein said at least on hydrophobic agent is a nanoparticle.

52. A method as in claim 51, wherein said nanoparticle is in a nanosuspension.

53. A method as in claim 51, wherein said at least one hydrophobic agent has size ranging from about 1 nm to about 1 micron.

54. A method for preparing a liposome comprising the step of using a hydrophobic agent nanosuspension as the aqueous phase of the liposome.

55. A method of preparing a multivesicular liposome comprising the step of using at least one hydrophobic agent nanosuspension as the first aqueous phase of a double emulsion process.

56. The method as in claim 55 wherein at least two different said hydrophobic agent nanosuspensions are used sequentially as first aqueous phases, whereby each agent is encapsulated in separate chambers.

57. A method for preparing a microsphere comprising the step of using a hydrophobic agent nanosuspension as the aqueous phase of the microsphere.

58. In a method for preparing a liposome, wherein the improvement comprises use of at least one hydrophobic agent nanosuspension as the aqueous component of the liposome.

59. In a method for preparing a mutivesicular liposome, wherein the improvement comprises use of at least one hydrophobic agent nanosuspension as the first aqueous component of the multivesicular liposome.

60. In a method for preparing a microsphere, wherein the improvement comprises use of at least one hydrophobic agent nanosuspension as the aqueous component of the microsphere.

61. A liposome produced by the method comprising the step of using at least one nanosuspension as the aqueous phase of the liposome.

62. A microsphere produced by the method comprising the step of using at least one nanosuspension as the aqueous phase of the microsphere.

63. A method for delivering at least one hydrophobic agent to a living being comprising injecting said living being with a composition comprising at least one nanoparticle encapsulated in a liposome.

64. A method for delivering at least one hydrophobic agent to a living being comprising injecting said living being with a composition comprising at least one nanoparticle encapsulated in a multivesicular liposome.

65. A method for delivering at least one hydrophobic agent to a living being comprising injecting said living being with a composition comprising at least one nanoparticle encapsulated in a microsphere.

66. A method for delivering at least one hydrophobic agent to a living being comprising administration to said living being of at least one nanoparticle encapsulated in a liposome via an inhalation device selected from the group consisting of nebulizer, metered dose inhaler, spray bottle, and intratracheal tube.

67. A method for delivering at least one hydrophobic agent to a living being comprising administration to said living being of at least one nanoparticle encapsulated in a microsphere via an inhalation device selected from the group consisting of nebulizer, metered dose inhaler, spray bottle, and intratracheal tube.
